(19) United States
(12) Patent Application Publication
(10) Pub. No.: US 2026/0051402 A1
(43) Pub. Date: Feb. 19, 2026

(54) ORIENTAL MEDICINE DIAGNOSIS AND PRESCRIPTION SYSTEM BASED ON ARTIFICIAL INTELLIGENCE AND OPERATION METHOD THEREOF

(71) Applicant: KEYherblab Co., Ltd., Gangwon-do (KR)

(72) Inventor: Euy Joon Roh, Gyeonggi-do (KR)

(21) Appl. No.: 18/912,829

(22) Filed: Oct. 11, 2024

(30) Foreign Application Priority Data
Aug. 16, 2024 (KR) 10-2024-0109863

(51) Int. Cl. G16H 50/20; G16H 10/20; G16H 20/10; G16H 20/90

(52) U.S. Cl. CPC G16H 50/20; G16H 10/20; G16H 20/10; G16H 20/90

(57) ABSTRACT

Disclosed is a herbal medicine diagnosis and prescription system which utilizes a deep learning model learned by using at least one of the information on oriental medicine formulations mapped to symptoms constituting the disclosed proof of evidence and information on personality types mapped to constitutional types comprises an artificial neural network based on a deep learning model which extracts the first and the second feature vectors from a user's questionnaire response data, receives the feature vectors as an input layer to extract at least one candidate oriental medicine formulation, sorts the extracted candidate oriental medicine formulations according to prescription priorities, or receives the second feature vector as an input layer to determine at least one health constitution type, and outputs health information mapped with the determined health constitution type. vacuum insulator layer laminated on the hot melt layer.

### CROSS-REFERENCE TO RELATED APPLICATION

This application claims priority of Korean Patent Application No. 10-2024-0109863, filed on Aug. 16, 2024, in the KIPO (Korean Intellectual Property Office), the disclosure of which is incorporated herein entirely by reference.

### BACKGROUND OF THE INVENTION

### Field of the Invention

The present invention relates to oriental medicine diagnosis and prescription technology, and more specifically, to an artificial intelligence-based oriental medicine diagnosis and prescription system and its operating method.

### Description of the Related Art

A number of hospitals are increasing every year, the number of oriental medicine doctors is also increasing, and the number of health insurance claims for oriental medicine formulations is also increasing. The oriental medicine doctors comprehensively analyze the information collected through the four diagnostic methods: visual inspection, auscultation, inquiry, and palpation, to gain a comprehensive understanding of the disease and make an oriental medicine diagnosis of the disease.

Based on these diagnoses, Korean medicine doctors treat patients through traditional Korean medicine practices passed down from ancestors, such as acupuncture, pharmacopuncture, moxibustion, cupping therapy, chuna therapy, herbal medicine, and oriental physical therapy, as well as modern applications and developments based on these traditional practices.

Recently, acupuncture-based prescriptions are being made due to the lack of specialized knowledge and clinical experience of oriental medicine doctors, and as a result of it, the frequency of oriental medicine formulation prescriptions is gradually decreasing. Even when oriental medicine formulation prescriptions are made, treatment is mainly based on simple symptoms. These prescriptions are based on traditional oriental medicine formulation prescriptions, and the prescriptions from the past were written in difficult Chinese characters, making it difficult to apply the prescriptions from the past in clinical practice. This lack of clinical experience may reduce the therapeutic effect.

On the other hand, since most diseases cause complex and complicated symptoms, it is very difficult for an unskilled oriental medicine doctor to accurately perform diagnosis. In addition, it is not easy for even an experienced oriental medicine doctor to accurately judge pattern identification due to the patient's subjective expression of symptoms, and inaccuracy of pattern identification may be a major factor in reducing the effectiveness of oriental medicine treatment.

In addition, since the subjective judgment of the oriental medicine doctor may be involved during a process finding pattern identification process, the same patient may experience different dialectical diagnosis depending on the case, which may ultimately lower the patient's trust in oriental medicine treatment.

In addition, although a number of health functional foods based on oriental medicine is increasing, psychological burden and time and economic costs are required due to busy lifestyle patterns and time and geographical conditions when using the health functional foods based on oriental medicine. Thus, it is difficult for office workers and people living in remote areas to actually use the health functional foods based on oriental medicine.

Therefore, even if oriental medicine doctors lack specialized knowledge of oriental medicine formulation, it is necessary to provide oriental medicine doctors with standardized and quantified prescription criteria by improving oriental medicine prescriptions, and a technology is also needed to make it easier for users to access oriental medicine-based health functional foods.

### SUMMARY OF THE INVENTION

The technological object to be achieved by the present invention is to provide an artificial intelligence-based oriental medicine diagnosis and prescription system and its operation method which improves oriental medicine prescriptions, provides Korean medicine doctors with standardized and quantified prescription criteria, and allows users to more easily access oriental medicine-based health functional foods.

In addition, the object of the present invention is to provide an AI-based oriental medicine diagnosis and prescription system which provides customized candidate oriental medicine formulations for each patient without side effects according to prescription priority.

The objects to be achieved by the present invention are not limited to the objects mentioned above, and other objects not mentioned may be understood by those skilled in the art from the description below.

According to one embodiment of the present invention, there is provided a traditional oriental medicine diagnosis and prescription system, a deep learning model learned using at least one of information on an oriental medicine formulation mapped to a symptom constituting a diagnosis and at least one of information on a personality type mapped to a constitutional type is utilized. The traditional oriental medicine diagnosis and prescription system may include an artificial neural network based on a deep learning model which extracts the first and the second feature vectors from a user's questionnaire response data, receives the feature vectors as an input layer to extract at least one candidate oriental medicine formulation, sorts the extracted candidate oriental medicine formulations according to prescription priorities, or receives the second feature vector as an input layer to determine at least one health constitution type, and outputs health information mapped with the determined health constitution type. The first feature vector may include information related to a cumulative score or probability value for symptoms constituting a disease symptom of the user and a diagnosis mapped to the candidate oriental medicine formulation, and the second feature vector may include health information related to a health constitution type of the user.

According to one embodiment, the artificial neural network may comprise: a first artificial neural network which outputs at least one candidate oriental medicine formulation defined by combination of at least one oriental medicine substance corresponding to the questionnaire response of the user, and at least the one oriental medicine substance; and a second artificial neural network which receives the candidate oriental medicine formulation output from the first neural network, accumulates questionnaire response scores related to symptoms constituting the diagnosis mapped to the candidate oriental medicine formulation, calculates a corresponding accumulated score or probability value of the symptom constituting the diagnosis, and deletes the symptom constituting the diagnosis mapped to the candidate oriental medicine formulation, so that he mapping relationship between the candidate oriental medicine formulation and the diagnosis may be updated if the corresponding accumulated score or probability value of the symptom constituting a diagnosis is lower than a threshold value, and determines the prescription priority of the candidate oriental medicine formulation in consideration of the updated mapping relationship. The second artificial neural network may maintain the mapping relationship between the candidate oriental medicine formulation and the symptom without updating if the cumulative score or probability value of the symptom constituting the diagnosis is greater than or equal to a threshold value.

The step for determining the prescription priority of the candidate oriental medicine formulations may include a first sorting based on the symptoms which determine a grade among symptoms constituting the diagnosis mapped to the candidate oriental medicine formulations, and a second sorting based on the symptoms which determine a rank among symptoms constituting the diagnosis mapped to the candidate oriental medicine formulations in the same grade.

The symptoms constituting the diagnosis include any one of symptoms of an essential symptom, a frequent symptom, a probable symptom, tendency and a new symptom, and the essential symptom, the frequent symptom, and the new symptom may be used as criteria for determining a rank, and the probable symptom, and tendency may be used as criteria for determining a rank in the same grade.

The artificial neural network may further include a third artificial neural network which outputs health information corresponding to the determined health constitution type by referring to a mapping table between the health constitution type and health information. The constitution type may include n types determined from a combination of temperature, eating, excretion, and mental.

According to another embodiment of the present invention, there is provided a traditional oriental medicine diagnosis and prescription server which utilizes a deep learning model learned by using at least one of the information on oriental medicine formulation mapped to symptoms constituting a diagnosis, and information on personality types mapped to constitutional types. The server is configured by a memory including one or more commands and one or more processors for executing the commands, and wherein the command includes commands for obtaining a user's questionnaire response data, extracting a feature vector from the user's questionnaire response data, determining at least one candidate oriental medicine formulation based on a first feature vector among the feature vectors, sorting the extracted candidate oriental medicine formulations according to prescription priority based on a second feature vector among the feature vectors, and outputting information about the sorted candidate oriental medicine formulations, the first feature vector may include information related to a disease symptom of the user, and the second feature vector may include information related to a cumulative score or a probability value for symptoms constituting a diagnosis mapped to the candidate oriental medicine formulation. The command may further include a command for determining at least one constitution type based on a third feature vector among above feature vectors, and outputting at least one personality type mapped to the determined constitution type. The command for determining at least one candidate oriental medicine formulation based on the first feature vector among the feature vectors may include a step for outputting at least one or more candidate oriental medicine formulations defined by at least one oriental medicine substance corresponding to the user's questionnaire response and a combination of the at least one oriental medicine substance.

A command for sorting the extracted candidate oriental medicine formulations according to prescription priority based on the second feature vector among the feature vectors accumulates questionnaire response scores related to the symptoms constituting the diagnosis mapped to the candidate oriental medicine formulations, and calculates the corresponding accumulated score or probability value of the symptoms constituting the diagnosis. If the cumulative score or the probability value of the symptom constituting the diagnosis is below a threshold, it may include a step for updating the mapping relationship between the candidate oriental medicine formulation and the diagnosis by deleting the symptom constituting the diagnosis mapped to the candidate oriental medicine formulation, and a step for determining the prescription priority of the candidate oriental medicine formulation in consideration of the updated mapping relationship. If the cumulative score or the probability value of the symptoms constituting the diagnosis is greater than or equal to a threshold, the mapping relationship between the candidate oriental medicine formulation and the diagnosis may be maintained without updating.

The command for determining the prescription priority of the candidate oriental medicine formulation may include a first sorting based on symptoms which determine a grade among symptoms constituting the diagnosis mapped to the candidate oriental medicine formulation, and a second sorting based on symptoms which determine a rank among symptoms constituting the diagnosis mapped to the candidate oriental medicine formulation in the same grade.

The constitution types may include n types determined from a combination of temperature, eating, excretion, and mental.

In a method for an operation method of a traditional oriental medicine diagnosis and prescription server which utilizes a deep learning model learned by using at least one of information on an oriental medicine formulation mapped to a symptom constituting a diagnosis and information on a personality type mapped to a constitution type, according to another embodiment of the present invention,

There may be provided an operation method of a server comprising: a step for obtaining a user's questionnaire response data; a step for extracting a feature vector from the user's questionnaire response data; a step for determining at least one candidate oriental medicine formulation based on a first feature vector among the feature vectors; a step for sorting the extracted candidate oriental medicine formulations according to a prescription priority based on a second feature vector among the feature vectors; and a step for outputting information about the sorted candidate oriental medicine formulations, and wherein the first feature vector includes information related to a disease symptom of the user, and the second feature vector includes information related to a cumulative score or a probability value for symptoms constituting a diagnosis mapped to the candidate oriental medicine formulation.

According to embodiments of the present invention, there is provided an AI-based oriental medicine diagnosis and prescription system, and an operation method thereof, which may provide standardized and quantified prescription criteria to oriental medicine doctors by improving the prescription of oriental medicine, and may allow users to access oriental medicine-based health functional foods more easily and conveniently by receiving the feature vector as an input layer to extract at least one candidate oriental medicine formulation, and sorting the extracted candidate oriental medicine formulation according to prescription priority, or by providing an artificial neural network based on a deep learning model that receives the second feature vector as an input layer, determines at least one constitution type, and selects at least one personality type mapped to the determined constitution type.

In addition, an AI-based oriental medicine diagnosis and prescription system which provides customized candidate oriental medicine formulations without side effects for each patient according to prescription priority may be provided.

In addition, an oriental medicine diagnosis and prescription server device and a method having the aforementioned advantages may be provided.

However, the effects of the present invention are not limited to the effects, and may be variously expanded within a scope which does not depart from the technological spirit and scope of the present invention.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a configuration diagram of an artificial intelligence-based oriental medicine diagnosis and prescription system according to an embodiment of the present invention.

FIG. 2 is a diagram illustrating a hardware configuration diagram of a server device that constitutes an artificial intelligence-based oriental medicine diagnosis and a prescription system according to an embodiment of the present invention.

FIG. 3A is a flowchart explaining an operation method of a server device for oriental medicine diagnosis and prescription according to an embodiment of the present invention. FIG. 3B is a flowchart explaining an operation method of a patient terminal for oriental medicine diagnosis and prescription.

FIG. 4 explains a deep learning model of an artificial intelligence-based oriental medicine diagnosis and prescription system according to an embodiment of the present invention.

FIG. 5 illustrates an example for sorting a plurality of extracted candidate oriental medicine formulations according to prescription priority according to an embodiment of the present invention.

FIG. 6 explains grading of symptoms constituting the indications of a candidate oriental medicine formulation according to an embodiment of the present invention.

FIG. 7 illustrates an example for distinguishing 16 health constitution types according to an embodiment of the present invention.

FIG. 8 shows an example of matching 16 constitution types and 16 health-related information according to an embodiment of the present invention.

In the following description, the same or similar elements are labeled with the same or similar reference numbers.

### DETAILED DESCRIPTION

Hereinafter, a preferred embodiment of the present disclosure will be elucidated in detail with reference to the accompanying drawings.

The embodiments of the present disclosure are provided for more completely explaining the present disclosure to those skilled in the art, the below embodiments can be modified to various forms and the scope of the present disclosure is not limited to the below embodiments. These embodiments are rather provided for more faithfully and completely explaining the present disclosure and for completely conveying the spirit of the present disclosure to those skilled in the art.

In the drawings, in addition, the dimension or thickness of each layer is exaggerated for clarity and convenience of the description and the same reference numeral indicates the same structural element. As used in the detail description, the term “and/or” includes any one of the listed items and one or more combination thereof. In addition, the term “connect” in the detail description means the state in which A member is directly connected to B member as well as the state in which C member disposed between A member and B member so that A member is indirectly connected to B member via C member.

The terms used herein are employed for describing the specific embodiment and the present disclosure is not limited thereto. As used in the detailed description and the appended claims, the singular forms may include the plural forms as well, unless the context clearly indicates otherwise. In addition, the terms “comprises” and/or “comprising” or “includes” and/or “including” used in the detailed description specify the presence of stated features, regions, integers, steps, operations, elements, and/or components, but do not preclude the presence or addition of one or more other features, regions, integers, steps, operations, elements, components, and/or groups thereof.

Although the terms of “first”, “second”, etc. are used herein to describe various members, parts, regions, layers and/or sections, it is obvious that these members, parts, regions, layers and/or sections should not be limited by the above terms. These terms are employed only for distinguishing one member, part, region, layer or section from another region, layer or section. Thus, the first member, the first part, the first region, the first layer or the first section described below may refer to the second member, the second part, the second region, the second layer or section without departing from the teachings of the present disclosure.

Furthermore, the terms related to a space such as “beneath”, “below”, “lower”, “above” and “upper” may be used to easily understand one element or a characteristic or another element or a characteristic illustrated in the drawings. The above terms related to the space are employed for easy understanding of the present disclosure depending on various process states or usage states of the present disclosure, and are not intended to limit the present disclosure.

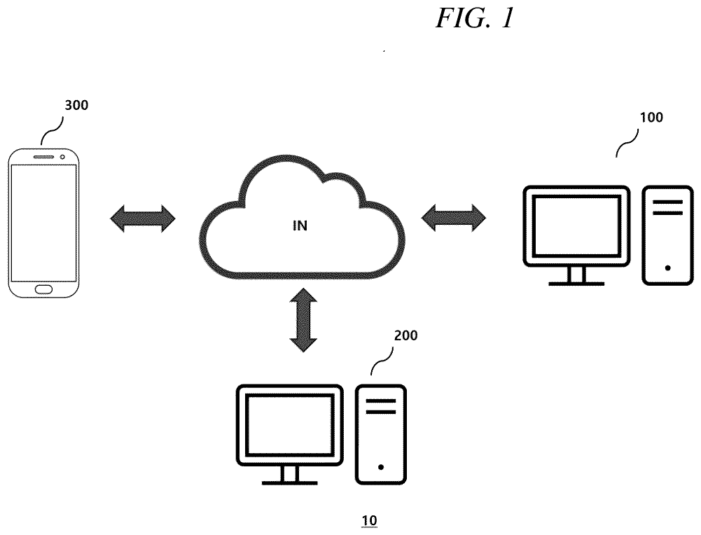

FIG. 1 is a configuration diagram of an artificial intelligence-based oriental medicine diagnosis and a prescription system according to an embodiment of the present invention.

The artificial intelligence-based oriental medicine diagnosis and prescription system 10 is composed of a patient terminal 300, an oriental medicine doctor terminal 200, and a server device 100, and the patient terminal 300, the oriental medicine doctor terminal 200, and the server device 100 may communicate with each other through a network (IN). Without limitation, the network (IN) may be implemented as any type of wired/wireless network, such as a local area network (LAN), a wide area network (WAN), a mobile radio communication network, a 3G or 4G network, or a 5G network.

In one embodiment, the patient terminal 300, the oriental medicine terminal 200, and the server device 100 may each be implemented as a single computing device. For example, the patient terminal 300 may be implemented as a first computing device, the oriental medicine terminal 200 may be implemented as a second computing device, and the server device 100 may be implemented as a third computing device. Alternatively, the first function of the artificial intelligence-based oriental medicine diagnosis and prescription system 10 may be implemented in a first computing device, the second function may be implemented in a second computing device, and the third function may be implemented in a third computing device. Alternatively, specific functions of the artificial intelligence-based oriental medicine diagnosis and prescription system 10 may be implemented in a plurality of computing devices.

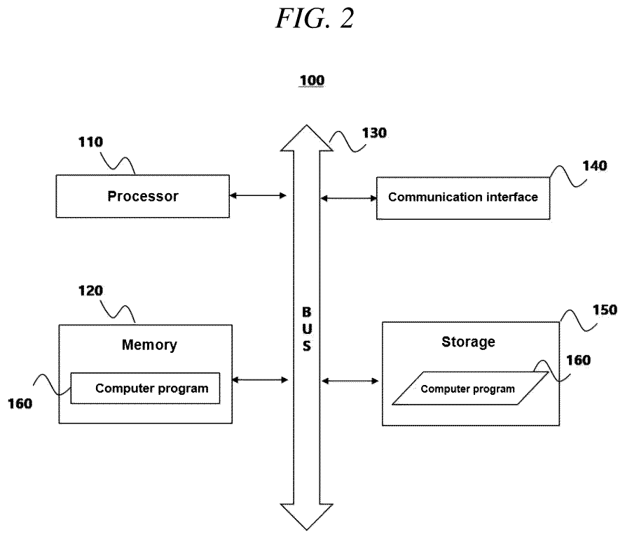

A computing device may encompass any devices having computing (processing) and communication capabilities, and FIG. 2 which will be described later is referred to with respect to an embodiment of such a device.

For your reference, as a computing device is a collection of various interacting components (e.g. memory, processor, etc.), it may be called a “computing system” in some cases. Of course, the term, a computing system may also mean a collection of interacting a plurality of computing devices.

The patient terminal 300 refers to a computing device used by a patient, and maybe, for example, a smart phone, laptop, desktop, or tablet PC owned by the patient. The oriental medicine doctor terminal 200 refers to a computing device used by an oriental medicine doctor, and may be a smart phone, laptop, desktop, or tablet PC owned by the oriental medicine doctor. The oriental medicine doctor may connect (access) to the server device 100 through the oriental medicine doctor terminal 200 and perform medical treatment. For example, if the server device 100 provides a web-based interface (e.g., a web page), the oriental medical doctor may perform medical treatment through a web browser installed on the oriental medical doctor terminal 200. That is, the oriental medicine doctor may perform a series of treatment processes through a web page that may add or modify the patient's medical questionnaire provided through the server device 100 and output candidate oriental medicine formulations extracted based on the deep learning model in order of prescription priority. That is, the oriental medicine doctor may also perform treatment through a treatment-related client application installed on the oriental medicine doctor's terminal 200. The oriental medicine doctor's terminal 200 may be implemented in any form.

The patient terminal 300 may refer to a computing device used by a patient. A patient may access the server device 100 through the patient terminal 300 to receive treatment. For example, a patient may access a webpage provided by the server device 100 through a web browser installed on the server device 100, receive an electronic questionnaire, and check the questionnaire items of the received electronic questionnaire to receive treatment. For example, a patient may receive prescription information by providing patient information and questionnaire response data corresponding to an electronic questionnaire through the web page. The patient terminal 300 may be implemented in any form. The questionnaire response data may be a short response or a plurality of answer responses which the patient checks for the questionnaire items in the electronic questionnaire, or a descriptive response which the patient answers for the questionnaire contents. The questionnaire items may be, but are not limited to, four-option a plurality of choice or five-option a plurality of choice. The descriptive response may be text data.

The server device 100 extracts a feature vector from response data for an electronic questionnaire of a patient or user (hereinafter referred to as “questionnaire response data”) based on the deep learning model described below, and determines a plurality of candidate oriental medicine formulations corresponding to the extracted feature vector. In addition, the above-determined candidate oriental medicine formulations may be sorted according to prescription priority and the result may be transmitted to the oriental medicine doctor terminal 200 or the patient terminal 300.

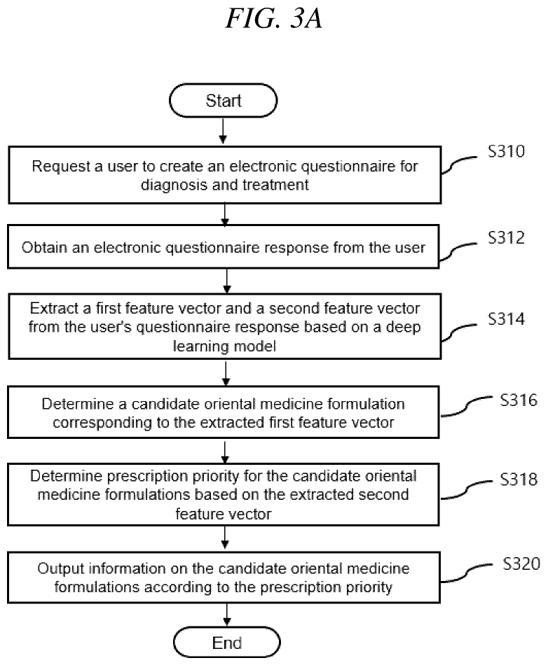

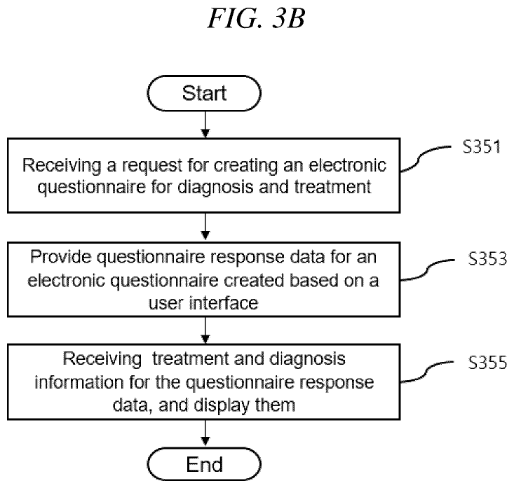

As described above, the operation of an artificial intelligence-based oriental medicine diagnosis and prescription system 10 according to some embodiments of the present invention has been briefly described with reference to FIG. 1. Hereinafter, the methods (i.e., detailed operations) that may be performed in an artificial intelligence-based oriental medicine diagnosis and prescription system 10 will be described in detail with reference to the drawings of FIGS. 3A and 3B. In the following description, if the subject of a specific step/an operation is omitted, it may be understood that it is performed in an artificial intelligence-based oriental medicine diagnosis and prescription system 10.

FIG. 2 illustrates a hardware configuration diagram of a server device that constitutes an artificial intelligence-based oriental medicine diagnosis and a prescription system according to an embodiment of the present invention.

FIG. 2 is a hardware configuration diagram illustrating a computing device 100, 200, 300.

As illustrated in FIG. 2, a computing device 100, 200, 300 may include one or processors 110, a bus 130, a communication interface 140, a memory 120 for loading a computer program 160 executed by the processor 110, and a storage 150 for storing the computer program 160. However, only components related to the embodiment of the present invention are illustrated in FIG. 2. Therefore, a person skilled in the art to which the present invention pertains may recognize that other general components may be included in addition to the components illustrated in FIG. 2. That is, the computing device 100, 200, 300 may further include various components in addition to the components illustrated in FIG. 2. In addition, in some cases, the computing device 100, 200, 300 may be configured as a form in which some of the components illustrated in FIG. 2 are omitted. Hereinafter, each component of the computing device 100, 200, 300 will be described.

The processor 110 may control the overall operation of each component of the computing device 100, 200, 300. The processor 110 may be configured to include at least one of a CPU (Central Processing Unit), an MPU (Micro Processor Unit), an MCU (Micro Controller Unit), a GPU (Graphics Processing Unit), or any other type of processor well known in the art of the present invention. Additionally, the processor 110 may perform operations for at least one application or program to execute operations/methods according to the embodiments of the present invention. The computing device 100, 200, and 300 may have one or more processors.

The memory 120 may store various data, commands, and/or information. The memory 120 may load a computer program 150 from the storage 150 to execute operations/methods according to embodiments of the present invention. For example, when a computer program (e.g., 160) related to the artificial intelligence-based oriental medicine diagnosis and prescription system 10 described above is loaded into the memory 120, functional logics such as those exemplified in FIG. 2 may be implemented on the memory 120. The memory (1202) may be implemented as a volatile memory such as RAM, but the technological scope of the present invention is not limited thereto.

The bus 130 may provide a communication function between the components of the computing device 100, 200, and 300. The bus 130 may be implemented as various types of buses such as an address bus, a data bus, and a control bus.

The communication interface 140 may support a wired and wireless Internet communication of the computing device 100, 200, 300. In addition, the communication interface 140 may support various communication methods other than Internet communication. To this end, the communication interface 140 may be configured to include a communication module well known in the technological field of the present invention.

The storage 150 may non-temporarily store one or more computer programs 226. The storage 150 may be configured to include nonvolatile memory such as Read Only Memory (ROM), Erasable Programmable ROM (EPROM), Electrically Erasable Programmable ROM (EEPROM), flash memory, a hard disk, a removable disk, or any form of computer-readable recording medium well known in the art to which the present invention pertains.

The computer program 160 may include one or more commands which cause the processor 110 to perform operations/methods according to various embodiments of the present invention when being loaded into the memory 120. That is, the processor 110 may perform operations/methods according to various embodiments of the present invention by executing the loaded one or more commands.

For example, the computer program 160 may include commands for executing an operation of obtaining a user's questionnaire response data, an operation of extracting a feature vector from the user's questionnaire response data, an operation of determining at least one candidate oriental medicine formulation based on a first feature vector among the feature vectors, an operation for sorting the extracted candidate oriental medicine formulations according to prescription priority based on the second feature vector among the feature vectors, and an operation for outputting information on the sorted candidate oriental medicine formulations, an operation of determining at least one health constitution type based on a third feature vector among the feature vectors, and an operation of generating health information mapped to the determined health constitution type. In such a case, an artificial intelligence-based oriental medicine diagnosis and prescription system 10 according to some embodiments of the present invention may be implemented through a computing device 100, 200, 300.

Meanwhile, in some embodiments, the computing device 100, 200, 300 illustrated in FIG. 2 may be a virtual machine implemented based on cloud technology. For example, the computing device 300 may be a virtual machine operating on one or more physical servers included in a server farm. In this case, at least some of the processor 110, memory 120, and storage 150 illustrated in FIG. 2 may be virtual hardware, and the communication interface 140 may also be implemented as a virtualized networking element such as a virtual switch.

As described above, an exemplary computing device 100, 200, 300 capable of implementing an artificial intelligence-based oriental medicine diagnosis and prescription system 10 according to some embodiments of the present invention has been described with reference to FIG. 2.

The technological concepts of the present invention described with reference to FIGS. 3 to 8 described below may be implemented as a computer-readable code on a computer-readable medium. The computer-readable recording medium may be, for example, a removable recording medium (CD, DVD, Blu-ray disc, USB storage device, removable hard disk) or a fixed recording medium (ROM, RAM, computer-attached hard disk). The computer program recorded on the computer-readable recording medium may be transmitted to another computing device through a network such as the Internet and installed on the other computing device, thereby allowing it to be used on the other computing device.

FIG. 3A and FIG. 3B are exemplary flowcharts illustrating a method for oriental medicine diagnosis and prescription according to some embodiments of the present invention. However, this is only a preferred embodiment for achieving the purpose of the present invention, and it is to be understood that some steps may be added or deleted as necessary.

FIG. 3A is a flowchart explaining an operation method of a server device for oriental medicine diagnosis and prescription according to an embodiment of the present invention, and FIG. 3B is a flowchart explaining an operation method of a patient terminal for oriental medicine diagnosis and prescription.

Referring to FIG. 3A, the server device 300 may perform oriental medicine diagnosis and prescription by utilizing a deep learning model learned by using at least one of information on an oriental medicine formulation mapped to a symptom constituting the diagnosis and information on a personality type mapped to a constitution type. The server device 300 may be composed of a memory including one or more commands, and one or more processors which execute the commands. The command may include a command to perform an operation S310 for requesting a user to create an electronic questionnaire for diagnosis and treatment, an operation S312 for obtaining the user's questionnaire response data, an operation for extracting a feature vector from the user's questionnaire response data, an operation for determining at least one candidate oriental medicine formulation based on a first feature vector among the feature vectors, an operation for sorting the extracted candidate oriental medicine formulations according to prescription priority based on a second feature vector among the feature vectors, and an operation for outputting information on the sorted candidate oriental medicine formulations.

The first feature vector is information related to the user's disease symptoms, and is used to predict the user's disease through the first feature vector. The second feature vector is information related to the cumulative scores or probability value of a proof of evidence mapped to the candidate oriental medicine formulation, and is used to sort the candidate oriental medicine formulations in order of prescription priority, which will be described later. The proof of evidence is diagnosis of the prescription, and the various symptoms constituting the proof of evidence may be classified into essential symptom, frequent symptom, probable symptom, and tendency. In other words, the above-mentioned proof of evidence may be classified into ‘essential symptom, frequent symptom, probable symptom, tendency and improper symptom’ based on the clinical probability symptom appearance frequency. The above-mentioned essential symptom is a symptom that must be present when prescribing, the above-mentioned frequent symptom is a symptom that frequently appear when prescribing, the above-mentioned probable symptom is a symptom that occasionally appear when prescribing, the tendency is a symptom that may appear when prescribing, and the improper symptom is a symptom that should not appear when prescribing. The various symptoms that constitute the proof of evidence may be confirmed through various means, including but not limited to, electronic questionnaires (pre- or post-questionnaires), the patient's voice, images of the patient's stool or tongue, etc. For example, an electronic questionnaire including questionnaire contents related to ‘essential symptoms, frequent symptoms, probable symptoms, tendencies, and improper symptoms’ may be transmitted to the patient terminal 300 described above, and the patient or the patient's guardian may fill out the electronic questionnaire, and the created electronic questionnaire (hereinafter referred to as questionnaire response data) may be provided to the server device 100.

In one embodiment, the command may further include a command causing the processor to perform an operation of determining at least one health constitution type based on a third feature vector among the feature vectors and an operation of generating health information mapped with the determined health constitution type.

The command for determining at least one candidate oriental medicine formulation based on a first feature vector among the feature vectors may include a step for performing an operation for outputting at least one candidate oriental medicine formulation defined by at least one oriental medicine formulation corresponding to the user's questionnaire response data, and a combination of the at least one oriental medicine formulation. The command for sorting the extracted candidate oriental medicine formulations according to the prescription priority based on the second feature vector among. The feature vectors may include a step for performing an operation for accumulating questionnaire response scores related to symptoms constituting the proof of evidence mapped to the candidate oriental medicine formulations to calculate a corresponding accumulated score or probability value of the symptom constituting the proof of evidence, an operation for step for deleting the symptom constituting the proof of evidence mapped to the candidate oriental medicine formulation when the corresponding accumulated score or probability value of the symptom constituting is lower than a threshold value, so that the mapping relationship between the candidate oriental medicine formulations and the proof of evidence may be updated, and an operation for determining prescription priority of the candidate oriental medicine formulations in consideration of the updated mapping relationship. If the cumulative score or probability value of the symptoms constituting the proof of evidence is greater than or equal to a threshold, the mapping relationship between the candidate oriental medicine formulation and the proof of evidence may be maintained without updating.

In one embodiment, the command for determining the prescription priority of the candidate oriental medicine formulation may include performing a first sorting operation based on a symptom to determine a grade among symptoms constituting the proof of evidence mapped to the candidate oriental medicine formulation, and a second sorting operation based a symptom to determine a rank in the same grade on among the symptoms constituting the proof of evidence mapped to the candidate oriental medicine formulation.

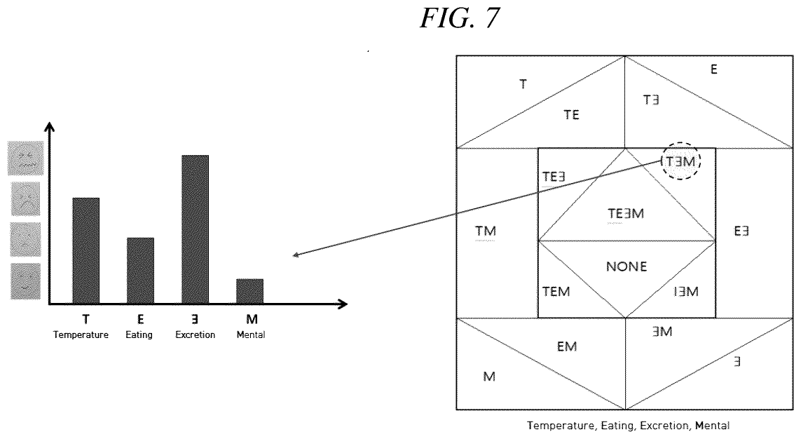

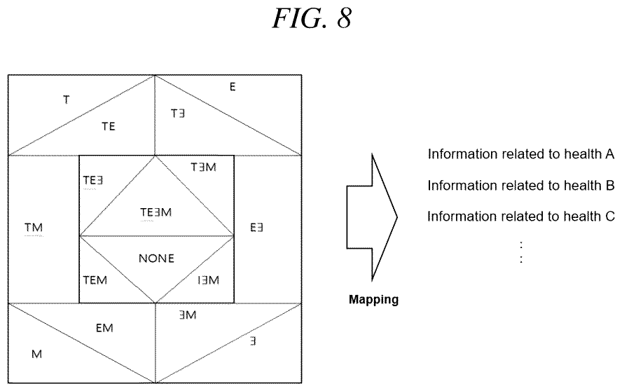

The health constitution type may include n types determined from a combination of temperature, eating, excretion, and mental. Refer to FIG. 7 and FIG. 8 for the health constitution type and the mapping relationship between the constitution type and personality type.

Meanwhile, the operation method of the oriental medicine diagnosis and prescription server 100 may include a step S312 for obtaining the user's questionnaire response data; a step S314 for extracting a feature vector from the user's questionnaire response data; a step S316 for determining at least one candidate oriental medicine formulation based on a first feature vector among the feature vectors; a step S318 for sorting the extracted candidate oriental medicine formulations according to prescription priority based on the second feature vector among the feature vectors; and a step S320 for outputting information on the sorted candidate oriental medicine formulations. Optionally, prior to the step S312, the server 100 may transmit a signal to the patient terminal 300 requesting the user to fill out an electronic questionnaire for diagnosis and treatment.

The oriental medicine diagnosis and prescription server 100 may optionally receive patient status feedback information, and use the patient status feedback information in the step S316 for determining a candidate oriental medicine formulation and/or the step S318 for sorting the candidate oriental medicine formulation according to prescription priority. The patient's progress may be rapidly improved by selecting a candidate oriental medicine formulation after considering the patient's progress through these steps. The patient condition feedback information may be treatment information entered after the oriental medicine doctor examines the patient's condition, or the patient's post-examination questionnaire response to the questionnaire written on the patient's smartphone regarding his or her condition. A post-examination questionnaire refers to a questionnaire used to check a patient's prognosis after the patient has been treated and received a prescription of oriental medicine formulation.

The operating method of the patient terminal 300 may include a step S351 for receiving a request for creating an electronic questionnaire for diagnosis and treatment from a server 100, a step S353 for providing questionnaire response data for an electronic questionnaire created by a user based on a user interface to the server 100, and a step S355 for receiving treatment and diagnosis information for the questionnaire response data, and displaying them. The above-mentioned diagnosis and treatment information may be health food information for the user or oriental medicine formulations for treating the user's disease symptoms. Alternatively, it may provide at least one of the user's constitution type and personality type.

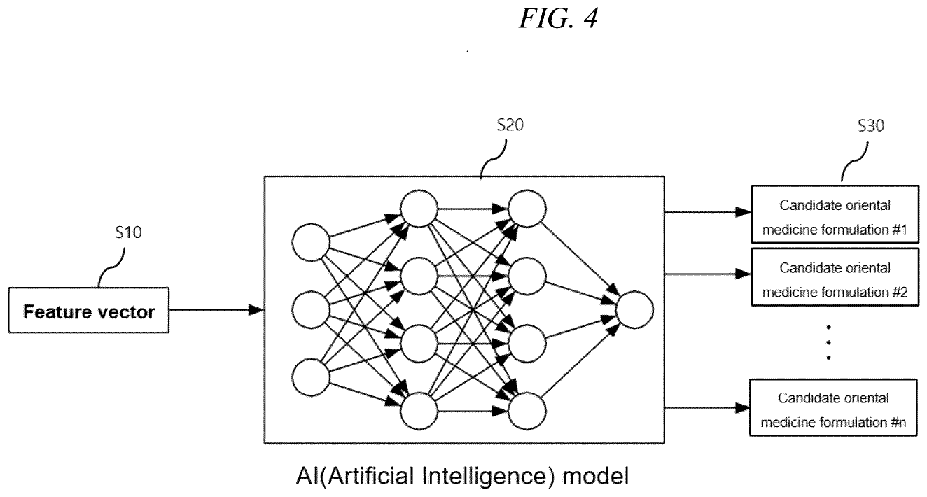

FIG. 4 is a diagram for explaining a deep learning model of an artificial intelligence-based oriental medicine diagnosis and prescription system 10 according to an embodiment of the present invention.

The deep learning model may be performed by the server device 100 described above. The deep learning model may be composed of a deep neural network (DNN), a convolutional neural network (CNN), a recurrent neural network (RNN), a restricted Boltzmann machine (RBM), a deep belief network (DBN), or a combination thereof. The deep neural network (DNN) is an artificial neural network which includes a plurality of hidden layers between the input layer and the output layer, and may learn various nonlinear relationships including a plurality of hidden layers. The convolutional neural network (CNN) is a type of multilayer perceptron designed to use minimal preprocessing, and consists of one or more convolutional layers and general artificial neural network layers placed on top of them, and additionally utilizes weights and layers. Due to this structure, CNN may fully utilize input data with a two-dimensional structure. A recurrent neural network (RNN) is a neural network in which the connections between the units that make up the artificial neural network form a directed circuit, and RNN may utilize the memory within the neural network to process arbitrary inputs. A restricted Boltzmann machine (RBM) is a model that removes intermediate connections from a Boltzmann machine. When connections between layers are removed, the machine takes the form of an undirected bipartite graph made up of visible units and hidden units. A deep belief neural network (DBN) is a graph-generating model used in machine learning. In deep learning, it refers to a deep neural network made up of a plurality of layers of latent variables. It has the characteristic that there are connections between layers, but no connections between units within a layer.

The deep learning model may be learned by using at least one of information about oriental medicine formulations mapped to symptoms constituting the proof of evidence based on the afore-mentioned neural network and information about personality types mapped to constitutional types. In addition, the deep learning model may be learned and improved through the clinical data of the patient. The clinical data should be used for learning the deep learning model without any legal problems as the patient consents to use it for clinical purposes.

First of all, the deep learning model may extract a feature vector from the questionnaire response data received from the user in order to predict an oriental medicine formulation to treat the user's disease symptoms (S10). Among the feature vectors, the first feature vector may include information related to a cumulative score or probability value for symptoms constituting the proof of evidence mapped to the user's disease symptoms and the candidate oriental medicine formulation, and the second feature vector may include information related to the user's constitution type.

The artificial neural network based on the deep learning model may receive feature vectors extracted from the user's questionnaire response data as an input layer, extract at least one candidate oriental medicine formulation, and sort the extracted candidate oriental medicine formulation according to prescription priority, or receive the second feature vector as an input layer, determine at least one constitution type, and select at least one personality type mapped to the determined constitution type (S20. S30).

In one embodiment, the artificial neural network may include a first artificial neural network that outputs at least one candidate oriental medicine formulation defined by at least one oriental medicine formulation corresponding to the user's questionnaire response, and a combination of the at least one oriental medicine formulation; and a second artificial neural network which receives the candidate oriental medicine formulation output from the first neural network as input, accumulates questionnaire response scores related to symptoms constituting the proof of evidence mapped to the candidate oriental medicine formulation, calculates the corresponding accumulated score or probability value of the symptom constituting the proof of evidence; and deletes the symptom constituting the proof of evidence mapped to the candidate oriental medicine formulation if the corresponding accumulated score or probability value of the symptom constituting the proof of evidence is below a threshold, the mapping relationship between the candidate oriental medicine formulation and the proof of evidence may be updated; and determines the prescription priority of the candidate oriental medicine formulation by considering the updated mapping relationship. The second artificial neural network maintains the mapping relationship between the candidate oriental medicine formulation and the symptom without updating if the corresponding cumulative score or probability value of the symptom constituting the proof of evidence is greater than or equal to a threshold, the mapping relationship between the candidate oriental medicine formulation formulations and the proof of evidence is maintained without updating. First of all, the determination of the prescription priority of the candidate oriental medicine formulations may be sorted based on symptoms that determine a grade among the symptoms constituting the proof of evidence mapped to the candidate oriental medicine formulation, and then, may be sorted based on symptoms which determine a rank among the symptoms constituting the proof of evidence mapped to the candidate oriental medicine formulation in the same grade. The symptoms constituting the proof of evidence include any one of basic symptom, anemia, edema, tendency, kidney syndrome, and basic symptom, anemia, and edema may be used as criteria for determining a grade, and edema and tendency may be used as criteria for determining a rank in the same grade.

In addition, the artificial neural network may further include a third artificial neural network that selects a personality type corresponding to the determined constitution type by referring to a mapping table between the constitution type and the personality type. The constitution type may include n types determined from a combination of temperature, eating, excretion, and mental.

In FIGS. 1 to 4, an example for extracting a feature vector based on an electronic questionnaire has been described, but the feature vector may be extracted by reflecting biometric information measured through a device that measures at least one of weight, body fat, body mass index (BMI), blood pressure, blood sugar, and sleep time, or a smartwatch worn on the user's wrist; health record information such as prescription medication and diagnosis records; and information on food and nutritional supplements currently being consumed, in addition to the electronic questionnaire.

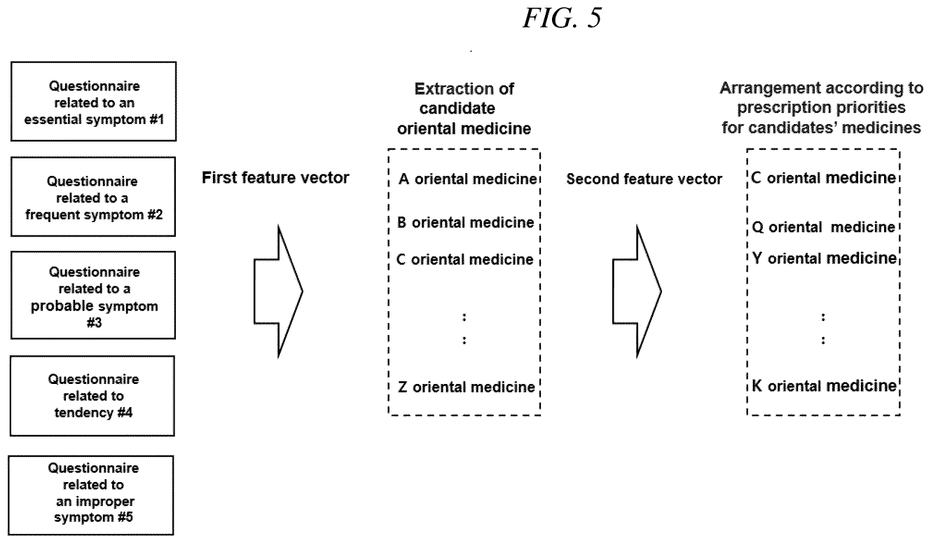

FIG. 5 is a diagram illustrating an example for sorting a plurality of extracted candidate oriental medicine formulations according to prescription priority according to an embodiment of the present invention.

Referring to FIG. 5, the questionnaire content in the electronic questionnaire described above may include questionnaire content for determining symptoms constituting proof of evidence, such as essential symptom, frequent symptom, probable symptom, tendency and improper symptom.

A plurality of candidate oriental medicine formulations (A to Z) may be extracted corresponding to the first feature vector extracted from the electronic questionnaire.

Since the first feature vector contains information related to the user's disease symptoms, the user's disease symptoms may be determined, and a plurality of candidate oriental medicine formulations which treat the determined disease symptoms may be determined. However, as the candidate oriental medicine formulation extracted by the first feature vector is obtained from the contents described in the oriental medicine formulation database or the oriental medicine classics, the oriental medicine formulation may be effective or may not improve the condition, in reality, depending on the patient's constitution, and the like.

In the present invention, based on the patient's questionnaire response data, a patient-tailored oriental medicine formulation service may be provided by sorting candidate oriental medicine formulations suitable for the patient in order of prescription priority. In another embodiment, it is necessary to prioritize and provide healthy foods that are suitable for the user based on oriental medicine formulations. The higher the prescription priority, the faster the patient's disease symptoms may be alleviated without side effects.

Specifically, the prescription priority of candidate oriental medicine formulations may be determined from the patient's response to the questionnaire to determine the presence of essential symptom, frequent symptom, probable symptom, tendency, and improper symptom. The priority ranking may be determined by grading based on essential symptom, frequent symptom, probable symptom, tendency, and improper symptom as follows.

1: a case that there are an essential symptom, a high frequent symptom, and a low frequent symptom

2: a case that there are no one or more low frequent symptoms

3: a case that there are no one or more high frequent symptoms

a: a case that there are one or more low new symptoms

b: a case that there is one or more a improper symptom

c: a case that there are one or more high improper symptoms

For example, as all of essential symptom, frequent symptom, probable symptom are appearing, and appearance of improper symptom is getting lower and lower, the rank may occupy high priority level.

The prescription priority may be determined according to the following order, that is, a case that the first candidate oriental medicine formulation has an essential symptom, a high frequent symptom, a low frequent symptom, and +one or more improper symptom (1a), a case that the second candidate oriental medicine formulation has both of an essential symptom, a high frequent symptom, but does not have at least one or more low frequent symptoms+at least one or more improper symptoms (2b), and a case that the third candidate oriental medicine formulation has all of the essential symptoms, does not have at least one or more high frequent symptoms+at least one or more improper symptoms (3c).

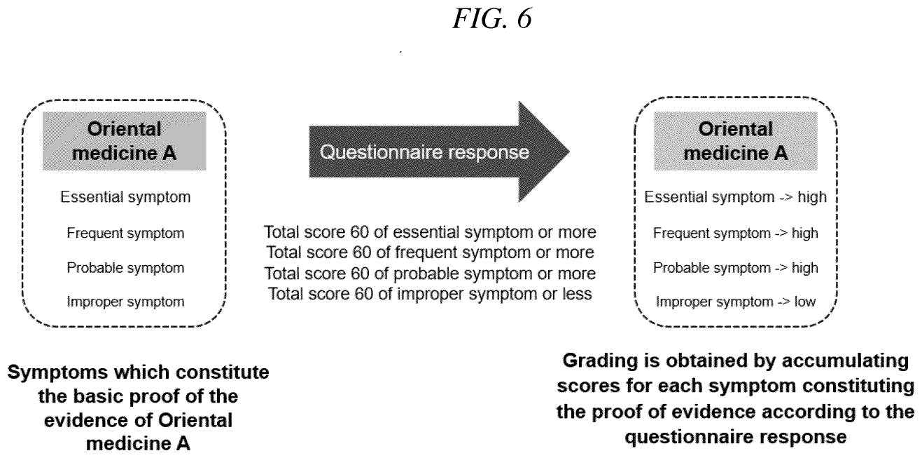

FIG. 6 is a diagram for explaining grading of symptoms constituting the proof of evidence of a candidate oriental medicine formulation according to an embodiment of the present invention.

Referring to FIG. 6, the oriental medicine formulations basically have the symptoms that constitute proof of evidence. For example, oriental medicine formulation which is extracted as a candidate oriental medicine formulation is generally used for treating an essential symptom, a frequent symptom, a probable symptom, and an improper symptom among the proof of evidence. After this, the oriental medicine formulation A which is generally used for treating an essential symptom, a frequent symptom, a probable symptom, and an improper symptom among the proof of evidence may judge a degree of an essential symptom, a frequent symptom, a probable symptom, and an improper symptom of a patient by calculating a cumulative score for each of an essential symptom, a frequent symptom, a probable symptom, and an improper symptom in order to determine the prescription priority, based on the user's questionnaire response. In another embodiment, it may be calculated as a probability value instead of a cumulative score. In this way, it is possible determine the priority between the oriental medicine formulation A and other oriental medicine formulations by judging whether a patient's essential symptoms are high/medium/low, whether a patient's frequent symptoms are low/medium/high, whether a patient's probable symptoms are high/medium/low, or whether a patient's improper symptoms are high/medium/low, and mapping them with the essential symptoms/the frequent symptoms/the probable symptoms/the improper symptoms which the oriental medicine formulation A basically has. The levels can be further subdivided than the high/medium/low levels. For example, it can be divided into nine levels: 1st, 2nd, 3rd high/1st, 2nd, 3rd medium/1st, 2nd, 3rd low.

FIG. 7 is a diagram illustrating an example for distinguishing 16 health constitution types according to an embodiment of the present invention.

Referring to FIG. 7, the user's health constitution type may be classified based on four scales of body temperature (T), eating (E), excretion (∃), and mental (M) among the questionnaire contents. A total of 16 health constitution types may be classified based on the four standards without limitation, and in some cases, this may be expanded to 64 constitution types. Specifically, it may be classified into a case that the user has difficulty only with temperature (T) (T), a case that the user has difficulty only with temperature (T) and eating (E) (TE), a case that the user has difficulty only with temperature (T) and excretion (∃) (T∃), a case that the user has difficulty only with eating (E) (E), a case that the user has difficulty only with temperature (T) and mind (M) (TM), a case that the user has difficulty only with eating (E) and excretion (∃) (E∃), a case that the user has difficulty only with temperature (T), eating (E), and excretion (∃) (TM∃), a case that the user has difficulty only with temperature (T), excretion (∃) and mind (M) (T∃M), a case that the user has difficulty only with temperature (T), eating (E), excretion (∃) and mind (M) (TE∃M), a case that the user has difficulty only with temperature (T), eating (E), excretion (∃) and mind (M) (TE∃M), Temperature (T), a case that the user has no difficulty with eating (E), excretion (∃), and mind (M) (NONE), a case that the user has difficulty only with temperature (T), eating (E), and mind (M) (TEM), a case that the user has difficulty only with eating (E), excretion (∃), and mind (M) (E∃M), a case that the user has difficulty only with eating (E) and excretion (∃) (E∃), a case that the user has difficulty only with mind (M) (M), a case that the user has difficulty only with eating (E) and mind (M) (EM), a case that the user has difficulty only with excretion (∃) and mind (M) (∃M), a case that the user has difficulty only with excretion (∃) (∃). FIG. 7 shows an example of a case (TIM) where a patient has difficulties with temperature (T), excretion (∃), and mind (M).

Although FIG. 7 explains the 16 types of health constitutions, the 16 types of health constitutions may be expanded into 64 types without limitation in three subdivisions by the symptoms that constitute the proof of evidence of the electronic questionnaire described above.

Each of these health constitution types may be mapped to corresponding health information, so that health information including at least one of the healthy lifestyle patterns/foods/exercises suitable for the user or patient may be created and mapped for each health constitution type. For example, information about what kind of healthy lifestyle a patient with a certain health constitution should have, what foods are beneficial and what foods are harmful, what kind of exercise to avoid, etc. may be mapped and provided to the user or patient.

FIG. 8 is a diagram illustrating an example wherein 16 constitution types and 16 health-related information are matched according to an embodiment of the present invention.

Referring to FIG. 8, 16 health constitution types classified based on the four standards of body temperature (T), eating (E), excretion (∃), and mentality (M) of FIG. 7 may be mapped to corresponding health-related information (A, B, C, . . . ) and provided to users or patients. That is, one of 16 health constitution types is determined through the electronic questionnaire of the user mentioned above, and a piece of health information related to the user's health constitution type may be provided to the user. Specifically, the health-related information may include information about one's constitution, information about current health status and predicted diseases (unforeseen diseases), information about health by life cycle, information about health by time zone of the day, information about biorhythm, information about body type (external appearance), information about personality (internal appearance), information about food and nutritional supplements, information about exercise, information about music, and so on.

In the present specification, the preferred embodiments of the present invention have been disclosed, and although specific terms are used, these are only used in a general sense to easily describe the technological contents of the present invention and to help the understanding of the present invention, and are not used to limit the scope of the present invention. It will be apparent to those of ordinary skill in the art to which the present invention pertains that other modifications based on the technological spirit of the present invention may be implemented in addition to the embodiments disclosed herein. It will be appreciated by those of ordinary skill in the art that an oriental medicine diagnosis and prescription system and its operation method according to the embodiments described with reference to FIGS. 1 to 8 may be variously substituted, changed and modified without departing from the spirit of the present invention. Therefore, the scope of the invention should not be determined by the described embodiments, but should be determined by the technological concepts described in the claims.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims.

The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. An oriental medicine diagnosis and prescription system wherein a deep learning model learned using at least one of information on an oriental medicine formulation mapped to a symptom constituting a proof of evidence and at least one of information on a personality type mapped to a constitutional type is utilized,
wherein the oriental medicine diagnosis and prescription system includes an artificial neural network based on a deep learning model which extracts the first and the second feature vectors from a user's questionnaire response data, receives the feature vectors as an input layer to extract at least one candidate oriental medicine formulation, sorts the extracted candidate oriental medicine formulations according to prescription priorities, or receives the second feature vector as an input layer to determine at least one health constitution type, and outputs health information mapped with the determined health constitution type; and
wherein the first feature vector includes information related to a cumulative score or probability value for a disease symptom of the user and symptoms constituting diagnosis mapped to the candidate oriental medicine formulation, and
wherein the second feature vector includes health information related to a health constitution type of the user.

2. The system of claim 1, wherein the artificial neural network comprises:
a first artificial neural network which outputs at least one candidate oriental medicine formulation defined by at least one oriental medicine substance corresponding to the questionnaire response of the user, and a combination of the at least one oriental medicine substance; and
a second artificial neural network which receives the candidate oriental medicine formulation output from the first neural network, accumulates questionnaire response scores related to symptoms constituting the proof of evidence mapped to the candidate oriental medicine formulation, calculates a corresponding accumulated score or probability value of the symptom constituting the proof of evidence, and deletes the symptom constituting the proof of evidence mapped to the candidate oriental medicine formulation, so that he mapping relationship between the candidate oriental medicine formulation and the proof of evidence updates if the corresponding accumulated score or probability value of the symptom constituting a proof of evidence is lower than a threshold value, and determines a prescription priority of the candidate oriental medicine formulation in consideration of the updated mapping relationship.

3. The system of claim 2, wherein the second artificial neural network maintains the mapping relationship between the candidate oriental medicine formulation and the symptom without updating if the cumulative score or probability value of the symptom constituting the proof of evidence is greater than or equal to a threshold value.

4. The system of claim 2, wherein determining the prescription priority of the candidate oriental medicine formulations includes:
a first sorting based on the symptoms which determine a grade among symptoms constituting the proof of evidence mapped to the candidate oriental medicine formulations, and
a second sorting based on the symptoms which determine a rank among symptoms constituting the proof of evidence mapped to the candidate oriental medicine formulations in the same grade.

5. The system of claim 1,
wherein the symptom constituting the proof of evidence includes any one of symptoms of an essential symptom, a frequent symptom, a probable symptom, tendency and an improper symptom,
wherein the essential symptom, the frequent symptom, and the improper symptom are used as criteria for determining a rank, and the probable symptom, and tendency are used as criteria for determining a rank in the same grade.

6. The system of claim 1, wherein the artificial neural network further includes a third artificial neural network which outputs health information corresponding to the determined health constitution type by referring to a mapping table between the health constitution type and health information.

7. The system of claim 6,
wherein the constitution type includes n types determined from a combination of temperature, eating, excretion, and mental,
wherein the health information includes at least one or more of information about your constitution, information about your current health status and predicted diseases (unforeseen diseases), information about your health by life cycle, information about your health by time zone of the day, information about your biorhythm, information about your body type (outward appearance), information about your personality (inward appearance), information about food and nutritional supplements, information about exercise, and information about music.

8. An oriental medicine diagnosis and prescription server which utilizes a deep learning model learned by using at least one of the information on oriental medicine formulation mapped to symptoms constituting a proof of evidence, and information on personality types mapped to constitutional types,
wherein the server is configured by a memory including one or more commands and one or more processors for executing the commands,
wherein the command includes commands for obtaining a user's questionnaire response data, extracting a feature vector from the user's questionnaire response data, determining at least one candidate oriental medicine formulation based on a first feature vector among the feature vectors, sorting the extracted candidate oriental medicine formulations according to prescription priority based on a second feature vector among the feature vectors, and outputting information about the sorted candidate oriental medicine formulations,
wherein the first feature vector includes information related to a disease symptom of the user,
wherein the second feature vector includes information related to a cumulative score or a probability value for symptoms constituting a proof of evidence mapped to the candidate oriental medicine formulation.

9. The server of claim 8, wherein the command further includes a command for determining at least one constitution type based on a third feature vector among above feature vectors, and outputting at least one personality type mapped to the determined constitution type.

10. The server of claim 8, wherein the command for determining at least one candidate oriental medicine formulation based on the first feature vector among the feature vectors includes outputting at least one or more candidate oriental medicine formulations defined by a combination of at least one oriental medicine substance corresponding to the user's questionnaire response and at least the one oriental medicine substance.

11. The server of claim 8, wherein a command for sorting the extracted candidate oriental medicine formulations according to prescription priority based on the second feature vector among the feature vectors includes calculating the corresponding accumulated score or probability value of the symptoms constituting the proof of evidence by accumulating questionnaire response scores related to the symptoms constituting the proof of evidence mapped to the candidate oriental medicine formulations, updating the mapping relationship between the candidate oriental medicine formulation and the proof of evidence by deleting the symptom constituting the proof of evidence mapped to the candidate oriental medicine formulation if the cumulative score or the probability value of the symptom constituting the proof of evidence is below a threshold, and determining the prescription priority of the candidate oriental medicine formulation in consideration of the updated mapping relationship.

12. The server of claim 11, wherein the mapping relationship between the candidate oriental medicine formulation and the proof of evidence is maintained without updating if the cumulative score or the probability value of the symptoms constituting the proof of evidence is greater than or equal to a threshold.

13. The server of claim 11, wherein the command for determining the prescription priority of the candidate oriental medicine formulation includes a first sorting based on symptoms which determine a grade among symptoms constituting the proof of evidence mapped to the candidate oriental medicine formulation, and a second sorting based on symptoms which determine a rank among symptoms constituting the proof of evidence mapped to the candidate oriental medicine formulation in the same grade.

14. The server of claim 9, wherein the constitution types includes n types determined from a combination of temperature, eating, excretion, and mental.

15. An operation of an oriental medicine diagnosis and prescription server which utilizes a deep learning model learned by using at least one of information on an oriental medicine formulation mapped to a symptom constituting a proof of evidence and information on a personality type mapped to a constitution type, and comprising:
obtaining a user's questionnaire response data;
extracting a feature vector from the user's questionnaire response data;
determining at least one candidate oriental medicine formulation based on a first feature vector among the feature vectors;
sorting the extracted candidate oriental medicine formulations according to a prescription priority based on a second feature vector among the feature vectors; and
outputting information about the sorted candidate oriental medicine formulations,
wherein the first feature vector includes information related to a disease symptom of the user,
wherein the second feature vector includes information related to a cumulative score or a probability value for symptoms constituting a proof of evidence mapped to the candidate oriental medicine formulation.
